(19) United States
(12) Patent Application Publication
(10) Pub. No.: US 2005/0208005 A1
(43) Pub. Date: Sep. 22, 2005

(54) Cosmetic composition comprising particles having a core-shell structure

(76) Inventor: Franck Giroud, Clichy (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/915,433

(22) Filed: Aug. 11, 2004

Related U.S. Application Data
(60) Provisional application No. 60/511,328, filed on Oct. 16, 2003.

(30) Foreign Application Priority Data
Aug. 11, 2003 (FR) 03 50420

(51) Int. Cl. A61K 7/06

(52) U.S. Cl. 424/70.1

(57) ABSTRACT

Disclosed herein is a cosmetic composition comprising, in a physiologically acceptable medium, at least one agent exhibiting a cosmetic activity and particles comprising a core and a solid shell bonded to the core via a noncovalent bond, the core comprising at least one metal, the solid shell comprising at least one inorganic material, and the size of the particles is less than or equal to 500 nm. Further disclosed herein is a cosmetic process for the treatment of keratinous substances, such as hair, and for contributing sheen to keratinous substances, such as hair, comprising applying to the keratinous substances the cosmetic composition disclosed herein.

This application claims benefit of U.S. Provisional Application No. 60/511,328, filed Oct. 16, 2003.

Disclosed herein is a cosmetic composition comprising particles having a core-shell structure.

Further disclosed herein is a cosmetic process for the treatment of keratinous substances, such as hair, for contributing sheen thereto, using the composition.

Even further disclosed herein is the use of the composition for contributing sheen to keratinous substances, such as the hair.

The technical field of the invention can be defined as that of cosmetic compositions, such as hair compositions and compositions for the skin or nails.

The use of metal particles has already been disclosed in various types of cosmetic make-up compositions.

For instance, European Patent Application Number EP-A-1 082 952 discloses make-up compositions, such as for the nails, comprising glass particles covered with a metal layer which make it possible to obtain a make-up exhibiting a sparkling and wear-resistant metallic appearance.

Further, European Patent Application Number EP-A-953 330 relates to the combination of two different compositions respectively comprising metal particles of a goniochromatic pigment type and a pigment of conventional type having one of the colors of the goniochromatic pigment for producing a make-up with a metallic effect which can vary according to the angle of observation and which can exhibit iridescent effects.

In addition, International Patent Application WO-A-02/03913 discloses nail varnish compositions comprising particles in the form of aluminium platelets present in an amount ranging from 0.4% to 0.75%, by weight and film-forming agents having high molecular weights for producing a make-up of mirror type, i.e., for instance, a make-up having not only the color of the aluminium but also a sheen and an ability to reflect the separate components of an object.

Metal particles have also been incorporated in hair compositions.

Thus, it is possible to contribute to the hair a better sheen than that contributed by fatty substances by incorporating metal nanoparticles, for example, silver nanoparticles, in hair compositions.

Such compositions are disclosed in European Patent Application Number EP-A-1 064 918.

However, it has been found that the sheen contributed by such compositions may fade very rapidly over time.

In another field, International Application Number WO-A-00/78282 discloses the use of silver nanoparticles with a size ranging from 1 nm to 50 nm as antimicrobial agent in curable silicone rubber compositions. However, WO-A-00/78282 does not appear to disclose the use of encapsulated nanoparticles.

A need therefore remains for a cosmetic composition, and for example, a hair cosmetic composition, comprising metal particles which can have a high sheen, wherein this sheen can be maintained over a long period of time without significant fading over the course of time.

There also exists a need for a cosmetic composition, such as a hair composition, which, while exhibiting a long lasting high sheen, is stable over time.

Disclosed herein is a cosmetic composition which can meet, inter alia, at least one of these needs.

The disclosure herein provides a cosmetic composition which does not exhibit the disadvantages, failings, limitations and inconveniences of the known compositions and which can solve at least one of the problems of the known compositions.

Disclosed herein is a cosmetic composition comprising, in a physiologically acceptable medium, at least one agent exhibiting a cosmetic activity and particles comprising a core and a solid shell bonded to the core via a noncovalent bond, wherein the core comprises at least one metal, the solid shell comprises an inorganic material, and the size of the particles is less than or equal to 500 nm.

Cosmetic compositions as described above comprising the specific particles incorporated in the compositions according to the disclosure, which can be defined by specific structures, specific constituents and specific particle sizes, have never been mentioned before.

Surprisingly, as a result of the incorporation in the compositions disclosed herein of these specific particles, which may be described as encapsulated metal nanoparticles, the compositions disclosed herein such as the hair compositions make it possible to obtain a high sheen immediately after application thereof, i.e., immediately after treatment of the keratinous substrate.

However, in contrast to the known compositions which comprise different metal particles from those incorporated in the compositions disclosed herein, i.e., non-encapsulated metal particles, the high sheen obtained with the compositions of the present disclosure can be retained for a prolonged period of time.

By way of example, this high sheen can be maintained for a period of time which can reach, for example, one month or more for hair treated with the compositions of the disclosure, whereas a known composition, such as that disclosed in European Patent Application Number EP-A-1 064 918, which discloses different particles from those included in the compositions disclosed herein, for example, non-encapsulated particles, loses all its sheen or reflectivity after a period of one month.

In addition to the retention of the sheen over time, the cosmetic compositions disclosed herein can exhibit a markedly better stability over time than that of the known compositions, for example European Patent Application Number EP-A-1 064 918, which comprises different metal particles from those included in the compositions of the present disclosure, for example, non-encapsulated metal particles.

It would appear, but without wishing to be committed to any theory, that the specific metal particles employed in the compositions disclosed herein can limit the aggregation of the metal nanoparticles in polar media, such as water and/or ethanol, and thus make it possible to obtain colloidal dispersions of high stability without phase separation.

In addition, the protection contributed by the shell of the at least one inorganic material can have the effect of preventing surface oxidation of the at least one metal constituting the particles under the action of external agents, whether these are agents present in the composition or agents with which the particles are liable to be in contact when the composition is applied, such as sebum, sweat, tears, atmospheric agents, and the like.

By preventing oxidation of the at least one metal of the particles, the loss in reflectivity of the metal and the loss in sheen which are the consequence of this oxidation can be avoided.

In one aspect of the present disclosure, a hair cosmetic composition, for example, a hair cosmetic composition for contributing sheen to the hair is disclosed.

Further disclosed herein is a cosmetic process for the treatment of keratinous substances, such as the hair, such as for contributing sheen to keratinous substances and for example, to the hair, comprising applying to the keratinous substances or fibers, the composition as described above.

Even further disclosed herein is the use of the composition as described above for contributing sheen to keratinous substances, such as the hair.

Even further disclosed herein is the use of the specific particles as described herein in a cosmetic composition for contributing sheen to keratinous substances or fibers, such as the hair.

The disclosure will now be described in more detail as follows.

The cosmetic compositions disclosed herein comprise at least one agent exhibiting a cosmetic activity or having a cosmetic effect.

The term “agent exhibiting a cosmetic activity” or “cosmetic active principle”, as used herein, means as any active compound having a cosmetic or dermatological activity or alternatively any compound capable of modifying the appearance, the feel and/or the physicochemical properties of keratinous substances, such as the hair.

The at least one agent exhibiting a cosmetic activity (the at least one cosmetic active principle) disclosed herein may generally be chosen from:
saccharides, oligosaccharides and polysaccharides which may be optionally hydrolyzed and modified,
amino acids, oligopeptides, peptides, proteins, which may be optionally hydrolyzed and modified, poly(amino acid)s and enzymes,
branched and unbranched fatty acids and alcohols,
animal, vegetable and mineral waxes,
ceramides and pseudoceramides,
hydroxylated organic acids,
UV screening agents,
antioxidants and agents for combating free radicals,
chelating agents,
antidandruff agents,
seborrhoea-regulating agents,
soothing agents,
cationic surfactants,
cationic and amphoteric polymers,
organomodified and non-organomodified silicones,
mineral, vegetable and animal oils,
polyisobutenes and poly(α-olefin)s,
esters,
soluble and dispersed anionic polymers,
soluble and dispersed non-ionic polymers,
reducing agents,
coloring agents and coloring materials, such as hair dyes,
foaming agents,
film-forming agents,
particles (other than the particles having a core-shell structure as disclosed herein),
and mixtures thereof.

The at least one agent exhibiting a cosmetic activity is present in an amount ranging from 0.001% to 10%, such as ranging from 0.01% to 5%, by weight relative to the total weight of the cosmetic composition.

Generally, the compounds of saccharide, oligosaccharide or polysaccharide which may be optionally hydrolyzed and/or modified, which can be used herein, are chosen from those which are described, for example, in “Encyclopedia of Chemical Technology, Kirk-Othmer, Third Edition, 1982, volume 3, pp. 896-900, and volume 15, pp. 439-458”, in “Polymers in Nature, by E. A. MacGregor and C. T. Greenwood, published by John Wiley & Sons, Chapter 6, pp. 240-328, 1980” and in Industrial Gums—Polysaccharides and their Derivatives, edited by Roy L. Whistler, Second Edition, published by Academic Press Inc.”

Mention may be made of exemplary saccharides, oligosaccharides or polysaccharides which may be optionally hydrolyzed and/or modified and which can be used herein, including, but not limited to, glucans, modified or unmodified starches (such as those resulting, for example, from cereals, such as wheat, maize and rice, from vegetables, such as yellow split peas, and from tubers, such as potatoes and manioc) which are different from starch betainate (starch as described above), amylose, amylopectin, glycogen, dextrans, β-glucans, celluloses and their derivatives (methylcelluloses, hydroxyalkylcelluloses, ethylhydroxyethylcelluloses and carboxymethylcelluloses), fructosans, inulin, levan, mannans, xylans, lignins, arabans, galactans, galacturonans, chitin, glucoronoxylans, arabinoxylans, xyloglucans, galactomannans, glucomannans, pectic acids and pectins, alginic acid and alginates, arabinogalactans, carrageenans, agars, glycosaminoglucans, gums arabic, gums tragacanth, ghatti gums, karaya gums, locust bean gums, guar gums and xanthan gums.

Mention may be made, of exemplary amino acids, for example, cysteine, lysine, alanine, N-phenylalanine, arginine, glycine, leucine, and mixtures thereof. Mention may also be made of exemplary oligopeptides, peptides and proteins which may be optionally hydrolyzed and/or optionally modified and which can be used herein, including, but not limited to, hydrolysates of wool or silk proteins, optionally modified, and plant proteins, such as wheat proteins.

Mention may be made of exemplary poly(amino acid)s which can be used, including, but not limited to, polylysine.

Mention may be made of exemplary enzymes which can be used, including, but not limited to, laccases, peroxidases, lipases, proteases, glycosidases, dextranases, uricases, and alkaline phosphatase.

Mention may be made or exemplary branched and unbranched fatty acids suitable to be used herein, including, but not limited to, C₈-C₃₀ carboxylic acids, such as palmitic acid, oleic acid, linoleic acid, myristic acid, stearic acid, lauric acid, and mixtures thereof. The fatty alcohols, which can be used herein, comprise, for example, C₈-C₃₀ alcohols, such as palmityl, oleyl, linoleyl, myristyl, stearyl, and lauryl alcohols.

A wax as used herein is a lipophilic compound, solid at ambient temperature (approximately 25° C.), with a reversible solid/liquid change of state, having a melting point of greater than approximately 40° C. and which can range up to 200° C., and exhibiting, in the solid state, an anisotropic crystalline arrangement. Generally, the size of the crystals of the wax is such that the crystals diffract and/or scatter light, conferring on the composition which comprises them a cloudy appearance which is more or less opaque. Bringing the wax to its melting point, it is possible to render it miscible with oils and to form a microscopically homogeneous mixture but, upon bringing the temperature of the mixture back to ambient temperature, a recrystallization of the wax from the oils of the mixture is obtained which is detectable microscopically and macroscopically (opalescence).

Mention may be made of exemplary waxes which can be used herein, including, but not limited to, waxes of animal origin, such as beeswax, spermaceti, lanolin wax and lanolin derivatives; vegetable waxes, such as carnauba wax, candelilla wax, ouricury wax, Japan wax, cocoa butter and cork fiber and sugarcane waxes; and mineral waxes, for example, paraffin wax, petrolatum wax, lignite wax, microcrystalline waxes, and ozokerites.

Mention may be made of exemplary ceramides, including, but not limited to, ceramides of the classes I, II, III and V according to the Downing classification, such as N-oleyldehydrosphingosine.

The hydroxylated organic acids are chosen from those well known and used in the art. Mention may, for example, be made of citric acid, lactic acid, tartaric acid, and malic acid.

Sunscreens, active in the UV-A and/or UV-B regions, which can be used herein, are those well known to a person skilled in the art. Mention may, for example, be made of dibenzoylmethane derivatives, such as 4-methyldibenzoylmethane, 4-isopropyldibenzoylmethane, 4-tert-butyldibenzoylmethane, 2,4-dimethyldibenzoylmethane and 4-tert-butyl4′-diisopropyldibenzoylmethane; p-aminobenzoic acid and its esters, such as 2-ethylhexyl p-dimethylaminobenzoate and N-propoxylated ethyl p-aminobenzoate; salicylates, such as triethanolamine salicylate, cinnamic acid esters, such as 2-ethylhexyl 4-methoxycinnamate and methyl diisopropylcinnamate; menthyl anthranilate; benzotriazole derivatives; triazine derivatives; β,β-diphenylacrylate derivatives, such as 2-ethylhexyl 2-cyano-3,3-diphenylacrylate and ethyl 2-cyano-3,3-diphenylacrylate; 2-phenylbenzimidazole-5-sulphonic acid and its salts; benzophenone derivatives; benzylidenecamphor derivatives; silicone-comprising screening agents; and the like.

Mention may be made of exemplary antioxidants and agents for combating free radicals, which can be used herein, including, but not limited to, ascorbic acid, ascorbylated compounds, such as ascorbyl dipalmitate, t-butylhydroquinone, polyphenols, such as phloroglucinol, sodium sulphite, erythorbic acid, and flavonoids.

Chelating agents can be chosen, for example, from EDTA (ethylenediaminetetraacetic acid) and its salts, such as disodium EDTA and dipotassium EDTA, phosphate-comprising compounds, such as sodium metaphosphate, sodium hexametaphosphate and tetrapotassium pyrophosphate, and phosphonic acids and their salts, such as the salts of ethylenediaminetetramethylenephosphonic acid.

Antidandruff agents are chosen, for example, from:
benzethonium chloride, benzalkonium chloride, chlorhexidine, chloramine-T, chloramine-B, 1,3-dibromo-5,5-dimethylhydantoin, 1,3-dichloro-5,5-dimethylhydantoin, 3-bromo-1-chloro-5,5-dimethylhydantoin and N-chlorosuccinimide;
1-hydroxy-2-pyridone derivatives, such as 1-hydroxy-4-methyl-2-pyridone, 1-hydroxy-6-methyl-2-pyridone and 1-hydroxy-4,6-dimethyl-2-pyridone;
trihalocarbamides;
triclosan;
azole-comprising compounds, such as climbazole, ketoconazole, clotrimazole, econazole, isoconazole and miconazole b;
antifungal polymers, such as amphotericin B and nystatin;
selenium sulphides;
sulphur in its various forms, cadmium sulphide, allantoin, coal and wood tars and their derivatives, for example, oil of cade, undecylenic acid, fumaric acid, and allylamines, such as terbinafine.

The antidandruff agents may also be used in the form of their addition salts with physiologically acceptable acids, for example, in the form of salts of sulphuric, nitric, thiocyanic, hydrochloric, hydrobromic, hydriodic, phosphoric, acetic, benzoic, glycolic, aceturic, succinic, nicotinic, tartaric, maleic, palmitic, methanesulphonic, propanoic, 2-oxopropanoic, propanedioic, 2-hydroxy-1,4-butanedioic, 3-phenyl-2-propenoic, α-hydroxybenzeneacetic, ethanesulphonic, 2-hydroxyethanesulphonic, 4-methylbenzenesulphonic, 4-amino-2-hydroxybenzoic, 2-phenoxybenzoic, 2-acetyloxybenzoic, picric, lactic, citric, malic and oxalic acids and of amino acids.

The antidandruff agents mentioned above can also, if appropriate, be used in the form of their addition salts with physiologically acceptable organic or inorganic bases. Examples of organic bases included, but are not limited to, alkanolamines with low molecular weights, such as ethanolamine, diethanolamine, N-ethylethanolamine, triethanolamine, diethylaminoethanol and 2-amino-2-methylpropanedione; nonvolatile bases, such as ethylenediamine, hexamethylenediamine, cyclohexylamine, benzylamine and N-methylpiperazine; quaternary ammonium hydroxides, for example trimethylbenzylammonium hydroxide; and guanidine and its derivatives, such as its alkylated derivatives. Examples of inorganic bases include, but not limited to, the salts of alkali metals, such as sodium or potassium; ammonium salts; the salts of alkaline earth metals, such as magnesium or calcium; or the salts of cationic di-, tri- or tetravalent metals, such as zinc, aluminum and zirconium. Alkanolamines, ethylenediamine and inorganic bases, such as the salts of alkali metals, can be used herein.

The seborrhoea-regulating agents, for example, succinylchitosan and poly-β-alanine can be used herein.

The soothing agents, for example, azulene and glycyrrhetinic acid can be used herein.

Cationic surfactants are those well known per se, such as salts of primary, secondary or tertiary fatty amines which are optionally polyoxyalkylenated; quaternary ammonium salts, such as tetraalkylammonium, alkylamidoalkyltrialkylammonium, trialkylbenzylammonium, trialkylhydroxyalkylammonium and alkylpyridinium chlorides and bromides; and imidazoline derivatives.

As used herein, the term “cationic polymer” means any polymer comprising cationic groups and/or groups which can be ionized to cationic groups.

Cationic polymers which can be used herein can be chosen from all those already known per se as improving the cosmetic properties of hair treated with detergent compositions, for example, those disclosed in European Patent Application Number EP-A-0 337 354 and in French Patent Nos. FR-A-2 270 846, 2 383 660, 2 598 611, 2 470 596 and 2 519 863.

The cationic polymers may be chosen from, for example, at least one amine group chosen from comprising primary, secondary, tertiary and quaternary amine groups which can either form part of the main polymer chain or be carried by a side substituent directly connected to the main chain.

The cationic polymers used in the present compositions generally have a number-average molecular mass ranging from 500 to 5×10⁶ approximately, such as ranging from 10³ to 3×10⁶ approximately.

Mention may be made of exemplary cationic polymers, including, but not limited to, polymers of the polyamine, polyaminoamide and poly(quaternary ammonium) type. These are art recognized products.

The polymers of the polyamine, polyaminoamide and poly(quaternary ammonium) type which can be used in the composition disclosed herein include those disclosed in French Patents Nos. 2 505 348 and 2 542 997. Mention may be made, among these polymers, of:
(1) homopolymers and copolymers derived from esters or amides of acrylic and methacrylic acid;
(2) cellulose ether derivatives comprising quaternary ammonium groups disclosed in French Patent No. 1 492 597;
(3) cationic cellulose derivatives, such as the copolymers of cellulose and the cellulose derivatives grafted with a water-soluble quaternary ammonium monomer and disclosed, for example, in U.S. Pat. No. 4,131,576, such as hydroxyalkylcelluloses, for example hydroxymethyl-, hydroxyethyl- and hydroxypropyl-celluloses, grafted, for example, with a methacryloylethyltrimethylammonium, methacrylamidopropyltrimethylammonium and dimethyldiallylammonium salt;
(4) the cationic polysaccharides disclosed, for example, in U.S. Pat. Nos. 3,589,578 and 4,031,307, such as guar gums comprising trialkylammonium cationic groups;
(5) polymers comprising piperazinyl units and at least one group chosen from divalent, straight- and branched-chain alkylene and hydroxyalkylene groups, optionally interrupted by at least one entity chosen from oxygen, sulphur and nitrogen atoms and by aromatic and heterocyclic rings, as well as the oxidation and/or quaternization products of these polymers. Such polymers are disclosed, for example, in French Patents Nos. 2 162 025 and 2 280 361;
(6) water-soluble polyaminoamides, such as those disclosed, for example, in French Patent Nos. 2 252 840 and 2 368 508;
(7) polyaminoamide derivatives, for example the adipic acid/dialkylaminohydroxyalkyldialkylenetriamine polymers wherein the alkyl group comprises from 1 to 4 carbon atoms such as methyl, ethyl and propyl groups and the alkylene group comprises from 1 to 4 carbon atoms such as an ethylene group. Such polymers are disclosed, for example, in French Patent No. 1 583 363;
(8) polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms. The molar ratio of polyalkylenepolyamine to dicarboxylic acid ranges from 0.8:1 to 1.4:1; wherein the polyaminoamide resulting therefrom being reacted with epichlorohydrin in a molar ratio of epichlorohydrin with respect to the secondary amine group of the polyaminoamide ranges from 0.5:1 to 1.8:1. Such polymers are disclosed, for example, in U.S. Pat. Nos. 3,227,615 and 2,961,347.
(9) cyclopolymers of alkyldiallylamine or of dialkyldiallylammonium, such as the homopolymer of dimethyldiallylammonium chloride and the copolymers of diallyldimethylammonium chloride and of acrylamide;
(10) the quaternary diammonium polymers exhibiting a number-average molecular mass ranging from 1,000 to 100,000, such as those disclosed, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020;
(11) polymers of poly(quaternary ammonium)s, such as those disclosed, for example, in European Patent Application No. EP-A-1 22 324;
(12) quaternary polymers of vinylpyrrolidone and of vinylimidazole, such as the products sold under the names Luviquat® FC 905, FC 550 and FC 370 by BASF;
(13) polyamines, such as Polyquart® H sold by Henkel, referenced under the name of “Polyethylene Glycol (15) Tallow Polyamine” in the CTFA dictionary;
(14) crosslinked polymers of methacryloyloxy(C₁-C₄)alkyltri(C₁-C₄)alkylammonium salts, such as those sold under the names of Salcare® SC 92, Salcare® SC 95 and Salcare® SC 96 by Allied Colloids; and
   mixtures thereof.

Other cationic polymers, which can be used herein, include cationic proteins, cationic protein hydrolyzates, polyalkyleneimines, for example, polyethyleneimines, polymers comprising vinylpyridine and vinylpyridinium units, condensates of polyamines and of epichlorohydrin, quaternary polyureylenes and chitin derivatives.

The amphoteric polymers, which can be used herein, may be chosen from polymers comprising B and C units distributed randomly in the polymer chain, wherein the B unit is a unit deriving from a monomer comprising at least one basic nitrogen atom and the C unit is a unit deriving from an acidic monomer comprising at least one group chosen from carboxyl and sulpho groups or else the B and C units can be chosen from groups deriving from zwitterionic carboxybetaine, and sulphobetaine monomers; the B and C units can also be chosen from a cationic polymer chain comprising at least one amine group chosen from primary, secondary, tertiary and quaternary amine groups, wherein at least one of the amine groups carries a carboxyl or sulpho group connected via a hydrocarbonaceous group, or else the B and C units form part of a chain of a polymer comprising a dicarboxyethylene unit, at least one of the carboxyl groups of which has been reacted with a polyamine comprises at least one primary or secondary amine groups.

Exemplary amphoteric polymers corresponding to the definition given above include, but are not limited to the following polymers:
(1) polymers resulting from the copolymerization of a monomer derived from a vinyl compound carrying at least one carboxyl group, such as acrylic acid, methacrylic acid, maleic acid and α-chloroacrylic acid, and of a basic monomer derived from a substituted vinyl compound comprising at least one basic atom, such as dialkylaminoalkyl methacrylates and acrylates and dialkylaminoalkylmethacrylamides and -acrylamides. Such compounds are disclosed in U.S. Pat. No. 3,836,537. Mention may also be made of the sodium acrylate/acrylamidopropyltrimethylammonium chloride copolymer sold under the name Polyquart® KE 3033 by Henkel.

The vinyl compound can also be a dialkyldiallylammonium salt, such as diethyldiallylammonium chloride.

Copolymers of acrylic acid and of the latter monomer are provided under the names Merquat® 280, Merquat® 295 and Merquat® Plus 3330 by Calgon.
2) polymers comprising units derived:
   a) from at least one monomer chosen from acrylamides and methacrylamides substituted on the nitrogen by an alkyl group,
   b) from at least one acidic comonomer comprising at least one reactive carboxyl groups, and
   c) from at least one basic comonomer, such as esters comprising at least one amine substituent chosen from primary, secondary, tertiary and quaternary amine substituents of acrylic and methacrylic acids and the quaternization product of dimethylaminoethyl methacrylate with dimethyl or diethyl sulphate.

For example, N-substituted acrylamides and methacrylamides disclosed herein may include groups wherein the alkyl groups comprise from 2 to 12 carbon atoms such as N-ethylacrylamide, N-tert-butylacrylamide, N-tert-octylacrylamide, N-octylacrylamide, N-decylacrylamide and N-dodecylacrylamide, and the corresponding methacrylamides.

The acidic comonomers are, for example, chosen from acrylic, methacrylic, crotonic, itaconic, maleic and fumaric acids and alkyl monoesters comprising from 1 to 4 carbon atoms of maleic or fumaric acids or anhydrides. The basic comonomers are, for example, chosen from aminoethyl, butylaminoethyl, N,N-dimethylaminoethyl and N-tert-butylaminoethyl methacrylates. The copolymers wherein the CTFA name (4th Ed., 1991) is octylacrylamide/acrylates/butylaminoethyl methacrylate copolymer, such as the products sold under the name Amphomer® or Lovocryl® 47 by National Starch can, for example, be used.
(3) partially or completely alkylated and crosslinked polyaminoamides derived from polyaminoamides of formula (II): [symbol]
   CO—R₁₀—CO-Z [symbol] (II)
   wherein R₁₀ is a divalent group derived from a saturated dicarboxylic acid, from an aliphatic mono- or dicarboxylic acid comprising an ethylenic double bond, from an ester of a lower alkanol having from 1 to 6 carbon atoms of these acids, or from a group derived from the addition of any one of the acids with a bisprimary or bis(secondary derived) amine, and Z is a group of a bisprimary, mono- and bissecondary polyalkylenepolyamines such as groups comprising:
   a) in an amount ranging from 60 mol % to 100 mol %, the group of formula (III):
   —NH [symbol] (CH₂)ₓ-NH [symbol] ₚ (III)
   wherein x=2 and p=2 or 3, optionally x=3 and p=2 wherein this group is derived from diethylenetriamine, triethylenetetraamine or dipropylenetriamine;
   b) in an amount ranging from 0 mol % to 40 mol %, the above group (III), wherein x=2 and p=1 and which derives from ethylenediamine, or the group deriving from piperazine: 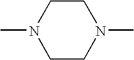
   c) in an amount ranging from 0 mol % to 20 mol %, the group —NH—(CH₂)₆—NH— deriving from hexamethylenediamine, wherein these polyaminoamides are crosslinked by addition of a bifunctional crosslinking agent chosen from epihalohydrins, diepoxides, dianhydrides and bisunsaturated derivatives, by means of from 0.025 mol to 0.35 mol of crosslinking agent per amine group of the polyaminoamide, and alkylated by reaction with acrylic acid, chloracetic acid or an alkanesultone or their salts.

The saturated carboxylic acids are, for example, chosen from acids having from 6 to 10 carbon atoms, such as adipic, 2,2,4-trimethyladipic and 2,4,4-trimethyladipic, and terephthalic acids, and the acids comprising an ethylenic double bond, such as, for example, acrylic, methacrylic and itaconic acids. The alkanesultones used in the alkylation are chosen from, for example, propane- and butanesultone and the salts of the alkylating agents are, for example, chosen from the sodium and potassium salts.
(4) polymers comprising zwitterionic units of formula (IV): 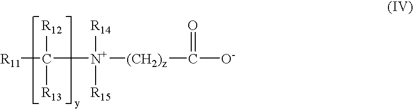
   wherein R₁₁ is a polymerizable unsaturated group, such as an acrylate, methacrylate, acrylamide or methacrylamide group, y and z are each an integer ranging from 1 to 3, R₁₂ and R₁₃, which may be identical or different, are chosen from hydrogen atoms and methyl, ethyl and propyl groups, and R₁₄ and R₁₅, which may be identical or different, are chosen from hydrogen atoms and alkyl groups such that the sum of the carbon atoms in R₁₄ and R₁₅ does not exceed 10.

The polymers comprising such units can also comprise units derived from non-zwitterionic monomers, such as dimethyl- and diethylaminoethyl acrylate and methacrylate, alkyl acrylates and methacrylates, acrylamides and methacrylamides, and vinyl acetate.

Mention may be made, by way of example, of the copolymer of methyl methacrylate and of dimethylcarboxymethylammonioethyl methacrylate, such as the product sold under the name Diaformer® Z301 by Sandoz.
(5) polymers derived from chitosan comprising monomer units corresponding to the following formulae: 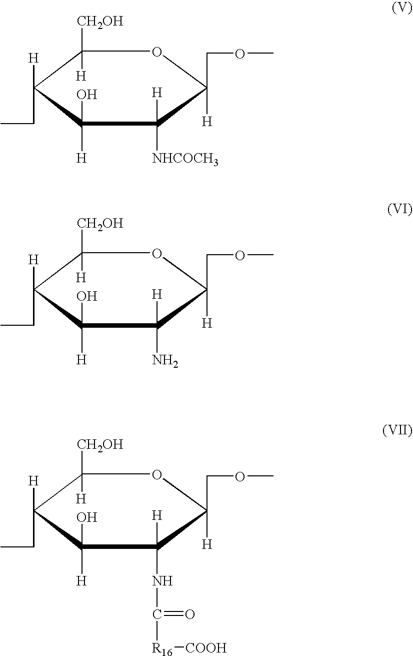
   wherein the unit (V) is present in an amount ranging from 0% to 30%, the unit (VI) is present in an amount ranging from 5% to 50% and the unit (VII) is present in an amount ranging from 30% and 90%, wherein in the unit (VII), R₁₆ is a group of formula (VIII): 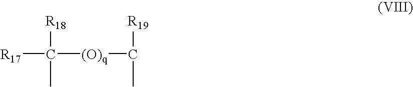
   wherein, if q=0, R₁₇, R₁₈ and R₁₉, which may be identical or different, are chosen from hydrogen atoms, methyl, hydroxyl, acetoxy and amino residues, monoalkylamino residues and dialkylamino residues, optionally interrupted by at least one nitrogen atom and optionally substituted by at least one group chosen from amino, hydroxyl, carboxyl, alkylthio and sulpho groups, or an alkylthio residues wherein the alkyl group carries an amino residue, wherein at least one of the R₁₇, R₁₈ and R₁₉ groups is, in this case, a hydrogen atom;
   optionally, if q=1, R₁₇, R₁₈ and R₁₉ are chosen from hydrogen atoms, and the salts formed by these compounds with bases or acids.
(6) polymers derived from the N-carboxyalkylation of chitosan, such as the N-(carboxymethyl)chitosan and the N-(carboxybutyl)chitosan sold under the name Evalsan® by Jan Dekker.
(7) polymers corresponding to the general formula (IX) disclosed, for example, in French Patent No. 1 400 366: 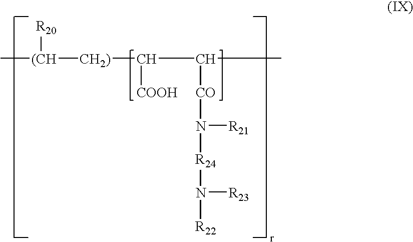
   wherein R₂₀ is chosen from a hydrogen atom and CH₃O, CH₃CH₂O and phenyl groups, R₂₁ is chosen from hydrogen and lower alkyl groups, such as methyl and ethyl, R₂₂ is chosen from hydrogen and lower alkyl groups, such as methyl and ethyl, and R₂₃ is chosen from lower alkyl groups, such as methyl and ethyl, and a group corresponding to the formula: —R₂₄—N(R₂₂)₂, R₂₄ is chosen from —CH₂—CH₂—, —CH₂—CH₂—CH₂— or —CH₂—CH(CH₃)— groups and R₂₂ has the meanings mentioned above, as well as the higher homologues of these groups comprising up to 6 carbon atoms.
(8) amphoteric polymers of the -D-X₁-D-X₁-type chosen from:
   a) polymers obtained by reaction of chloroacetic acid or sodium chloroacetate with compounds comprising at least one unit of formula (X):
   -D-X₁-D-X₁-D- (X)
   wherein D is a group 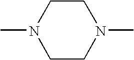
   and X₁ is chosen from the symbols E and E′, wherein E and E′, which may be identical or different, are chosen from bivalent alkylene groups comprising at least one chain chosen form straight- and branched-chain comprising up to 7 carbon atoms in the main chain, wherein the bivalent alkylene groups are optionally substituted by at least one hydroxyl group. E and E′ can additionally comprise at least one entity chosen from oxygen, nitrogen and sulphur atoms and 1 to 3 aromatic and heterocyclic rings; the oxygen, nitrogen and sulphur atoms being present in a form chosen from ether, thioether, sulphoxide, sulphone, sulphonium, alkylamine and alkenylamine groups and hydroxyl, benzylamine, amine oxide, quaternary ammonium, amide, imide, alcohol, ester and urethane groups.
   b) polymers of formula (XI):
   -D-X₁-D-X₁- (XI)
   wherein D is a group 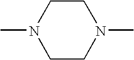
   and X₁ is chosen from the symbols E and E′, wherein at least one X₁ is chosen from E′, E having the meaning indicated above and E′ being chosen from bivalent alkylene groups comprising at least one chain from straight- and branched-chains comprising up to 7 carbon atoms in the main chain, wherein the bivalent alkylene groups are optionally substituted by at least one hydroxyl group. E′ can also comprise at least one nitrogen atom substituted by an alkyl chain optionally interrupted by an oxygen atom, wherein the alkyl chain comprises at least one functional group chosen from carboxyl functional groups at least one hydroxyl functional groups and wherein the alkyl chain is betainized by reaction with a reactant chosen from chloroacetic acid and sodium chloroacetate.
(9) (C₁-C₅)alkyl vinyl ether/maleic anhydride copolymers partially modified by semiamidation with an N,N-dialkylaminoalkylamine, such as N,N-dimethylamino-propylamine, or by semiesterification with an N,N-dialkanolamine. These copolymers can also comprise other vinyl comonomers, such as vinylcaprolactam.

The silicones which can be used herein can be soluble or insoluble in water and they can, for example, be polyorganosiloxanes which are insoluble in water; the silicones can be provided in the form of oils, of waxes, of resins or of gums.

Organopolysiloxanes are defined in more detail in the work by Walter Noll, “Chemistry and Technology of Silicones” (1968), Academic Press. They can be volatile or non-volatile.

When they are volatile, the silicones can be chosen from those having a boiling point ranging from 60° C. to 260° C. and such as from cyclic silicones comprising from 3 to 7 silicon atoms such as 4 to 5. They are, for example, octamethylcyclotetrasiloxane, sold, for example, under the name of “Volatile Silicone 7207” by Union Carbide or “Silbione 70045 V 2” by Rhodia, or decamethylcyclopentasiloxane, sold under the name of “Volatile Silicone 7158” by Union Carbide or “Silbione 70045 V 5” by Rhodia.

Mention may also be made of cyclocopolymers of the dimethylsiloxane/methylalkylsiloxane type, such as “Silicone Volatile FZ 3109”, sold by Union Carbide, with the chemical structure: 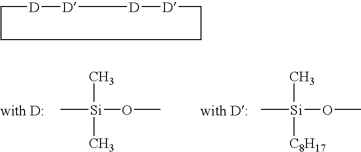
(ii) linear volatile silicones comprising from 2 to 9 silicon atoms and having a viscosity of less than or equal to 5×10⁻⁶ m²/s at 25° C. An example is decamethyltetrasiloxane, sold, for example, under the name “SH 200” by Toray Silicone. Silicones coming within this class are also described in the article published in Cosmetics and Toiletries, Vol. 91, January 76, p. 27-32, Todd & Byers, “Volatile Silicone Fluids for Cosmetics”.

Mention may, for example, be made, among nonvolatile silicones, of polyalkylsiloxanes, polyarylsiloxanes, polyalkylarylsiloxanes, silicone gums and resins, and polyorganosiloxanes modified by organofunctional groups.

The organomodified silicones which can be used herein are silicones as defined above and comprising, in their structure, at least one organofunctional groups attached via a hydrocarbonaceous group.

Mention may be made, among the organomodified silicones, of polyorganosiloxanes comprising:
at least one group chosen from polyethyleneoxy and polypropyleneoxy groups optionally comprising C₆-C₂₄ alkyl groups, such as the products known as dimethicone copolyol sold by Dow Corning under the name DC 1248 or the Silwet® L 722, L 7500, L 77 and L 711 oils from Union Carbide and the (C₁₂)alkyl methicone copolyol sold by Dow Corning under the name Q2 5200;
at least one amino group optionally substituted, such as the products sold under the names GP 4 Silicone Fluid and GP 7100 by Genesee and the products sold under the names Q2 8220 and Dow Corning 929 or 939 by Dow Corning. The substituted amino groups are, for example, chosen from C₁-C₄ aminoalkyl groups;
thiol groups, such as the products sold under the names “GP 72 A” and “GP 71” from Genesee;
alkoxylated groups, such as the product sold under the names “Silicone Copolymer F-755” by SWS Silicones and Abil Wax® 2428, 2434 and 2440 by Goldschmidt;
hydroxylated groups, such as the polyorganosiloxanes comprising a hydroxyalkyl functional group disclosed in French Patent Application No. FR-A-85 16334;
acyloxyalkyl groups, such as the polyorganosiloxanes disclosed in U.S. Pat. No. 4,957,732;
anionic groups of the carboxylic acid type, such as in the products disclosed in European Patent No. EP 186 507 from Chisso Corporation, or of the alkylcarboxylic type, such as those present in the product X-22-3701E from Shin-Etsu; 2-hydroxyalkylsulphonate and 2-hydroxyalkyl thiosulphate, such as the products sold by Goldschmidt under the names “Abil® S201” and “Abil® S255”;
hydroxyacylamino groups, such as the polyorganosiloxanes disclosed in European Patent Application No. EP 342 834. Mention may be made, for example, of the product Q2-8413 from Dow Corning.

Mention may be made of exemplary oils of vegetable origin, including, but not limited to, sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, maize oil, hazelnut oil, karite butter, palm oil, apricot kernel oil and calophyllum oil; as oils of animal origin, of perhydrosqualene; as oils of mineral origin, of liquid paraffin and liquid petrolatum.

The polyisobutenes and poly(α-olefin)s are chosen from those well known in the art.

Mention may be made of exemplary esters, including, but not limited to, esters of fatty acids, such as isopropyl myristate, isopropyl palmitate, 2-ethylhexyl palmitate, purcellin oil (stearyl octanoate), isononyl isononanoate, isostearyl isononanoate, isopropyl lanolate, and mixtures thereof.

The anionic polymers generally used herein are polymers comprising groups derived from acids chosen from carboxylic, sulphonic, and phosphoric acids and exhibiting a weight-average molecular mass ranging from 500 to 5,000,000.

The carboxyl groups are contributed by unsaturated carboxylic monoacid or diacid monomers, such as those corresponding to the formula (XII): 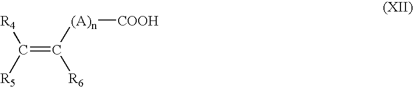
wherein n is an integer ranging from 0 to 10, A is a methylene group, optionally connected to the carbon atom of the unsaturated group or to the neighbouring methylene group when n is greater than 1 via a heteroatom, such as oxygen or sulphur, R₄ is chosen from hydrogen atoms and phenyl and benzyl groups, R₅ is chosen from hydrogen atoms and lower alkyls and carboxyl groups, and R₆ is chosen from hydrogen atoms, lower alkyl groups and —CH₂—COOH, phenyl and benzyl groups.

In the above formula (XII), the lower alkyl group, for example, comprises from 1 to 4 carbon atoms, such as the methyl and ethyl groups.

In one aspect of the present disclosure, the anionic polymers comprising carboxyl groups herein are chosen from:
A) Homo- or copolymers of acrylic or methacrylic acid or their salts such as the products sold under the names Versicol® E or K by Allied Colloid or Ultrahold® by BASF, the copolymers of acrylic acid and of acrylamide sold in the form of their sodium salt under the names Reten® 421, 423 or 425 by Hercules or the sodium salts of polyhydroxycarboxylic acids.
B) Copolymers of acrylic acid or methacrylic acid with a monoethylenic monomer, such as ethylene, styrene, vinyl esters and esters of acrylic and methacrylic acid, optionally grafted onto a polyalkylene glycol, such as polyethylene glycol, and optionally crosslinked. Such polymers are disclosed, for example, in French Patent No. 1 222 944 and German Patent Application No. 2 330 956, the copolymers of this type comprising, in their chain, an optionally N-alkylated and/or -hydroxyalkylated acrylamide unit, such as disclosed, for example, in Luxembourgian Patent Application Nos. 75370 and 75371 and provided under the name Quadramer® by American Cyanamid. Mention may also be made of copolymers of acrylic acid and of C₁-C₄ alkyl methacrylate and the copolymer of methacrylic acid and of ethyl acrylate sold under the name Luvimer® MAEX by BASF.
C) Copolymers derived from crotonic acid, such as those comprising, in their chain, vinyl acetate or propionate units and optionally other monomers, such as allyl and methallyl ester, vinyl ether and vinyl esters of a linear and branched saturated carboxylic acid comprising a long hydrocarbonaceous chain, such as those comprising at least 5 carbon atoms, it optionally being possible for these polymers to be grafted and crosslinked, or alternatively a vinyl, allyl or methallyl ester of an α- or β-cyclic carboxylic acid. Such polymers are disclosed, inter alia, in French Patent Nos. 1 222 944, 1 580 545, 2 265 782, 2 265 781, 1 564 110, and 2 439 798. Commercial products coming within this class are the Resins 28-29-30, 26-13-14 and 28-13-10 sold by National Starch.
D) Polymers derived from maleic, fumaric or itaconic acids or anhydrides with vinyl esters, vinyl ethers, vinyl halides, phenylvinyl derivatives, or acrylic acid and its esters; these polymers can be esterified. Such polymers are disclosed, for example, in U.S. Pat. Nos. 2,047,398, 2,723,248, and 2,102,113 and Patent No. GB 839 805 such as those sold under the names Gantrez® AN and ES by ISP.

Polymers also coming within this class are copolymers of maleic, citraconic or itaconic anhydrides and of an allyl or methallyl ester, optionally comprising an acrylamide or methacrylamide group, an α-olefin, acrylic or methacrylic esters, acrylic or methacrylic acids, or vinylpyrrolidone in their chain; the anhydride functional groups are monoesterified or monoamidated. These polymers are, for example, disclosed in French Patent Nos. 2 350 384 and 2 357 241.
E) Polyacrylamides comprising carboxylate groups.

The polymers comprising sulpho groups are polymers comprising vinylsulphonic, styrenesulphonic, naphthalenesulphonic or acrylamidoalkylsulphonic units.

These polymers can, for example, be chosen from:
salts of polyvinylsulphonic acid having a molecular mass of ranging from 1,000 and 100,000, as well as copolymers with an unsaturated comonomer, such as acrylic and methacrylic acids and their esters, as well as acrylamide and its derivatives, vinyl ethers and vinylpyrrolidone;
salts of polystyrenesulphonic acid, the sodium salts having a molecular mass of approximately 500 000 and of approximately 100 000 sold respectively under the names Flexan® 500 and Flexan® 130 by National Starch. These compounds are disclosed in French Patent No. 2 198 719;
salts of polyacrylamidosulphonic acids, such as those mentioned in U.S. Pat. No. 4,128,631 such as the polyacrylamidoethylpropanesulphonic acid sold under the name Cosmedia Polymer® HSP 1180 by Henkel.

As disclosed herein, the anionic polymers are, for example, chosen from acrylic acid copolymers, such as the acrylic acid/ethyl acrylate/N-tert-butylacrylamide terpolymer sold under the name Ultrahold Strong® by BASF; copolymers derived from crotonic acid, such as the vinyl acetate/vinyl tert-butylbenzoate/crotonic acid terpolymers and the crotonic acid/vinyl acetate/vinyl neododecanoate terpolymers sold under the name Resin 28-29-30 by National Starch; polymers derived from maleic, fumaric and itaconic acids and anhydrides with vinyl esters, vinyl ethers, vinyl halides, phenylvinyl derivatives, and acrylic acid and its esters, such as the monoesterified methyl vinyl ether/maleic anhydride copolymer sold under the name Gantrez® ES 425 by ISP; copolymers of methacrylic acid and of methyl methacrylate sold under the name Eudragit® L by Rohm Pharma; the copolymer of methacrylic acid and of ethyl acrylate sold under the name Luvimer® MAEX by BASF; the vinyl acetate/crotonic acid copolymer sold under the name Luviset® CA 66 by BASF; and the vinyl acetate/crotonic acid/polyethylene glycol terpolymer sold under the name Aristoflex® A by BASF.

As disclosed herein, the anionic polymers can also be used in the latex or pseudolatex form, i.e., in the form of an aqueous dispersion of insoluble polymer particles.

Mention may, for example, be made, as non-ionic polymers which can be used herein, of:
vinylpyrrolidone homopolymers;
copolymers of vinylpyrrolidone and of vinyl acetate;
polyalkyloxazolines, such as the polyethyloxazolines provided by Dow Chemical under the names PEOX® 50 000, PEOX® 200 000 and PEOX® 500 000;
vinyl acetate homopolymers, such as the product provided under the name Appretan® EM by Hoechst and the product provided under the name Rhodopas® A 012 by Rhône-Poulenc;
copolymers of vinyl acetate and of acrylic ester, such as the product provided under the name Rhodopas® AD 310 from Rhône-Poulenc;
copolymers of vinyl acetate and of ethylene, such as the product provided under the name Appretan® TV by Hoechst;
copolymers of vinyl acetate and of maleic ester, for example of dibutyl maleate, such as the product provided under the name Appretan® MB Extra by Hoechst;
copolymers of polyethylene and of maleic anhydride;
alkyl acrylate homopolymers and alkyl methacrylate homopolymers, such as the product provided under the name Micropearl® RQ 750 by Matsumoto and the product provided under the name Luhydran® A 848 S by BASF;
acrylic ester copolymers, such as copolymers of alkyl acrylates and of alkyl methacrylates, such as the products provided by Rohm & Haas under the names Primal® AC-261 K and Eudragit® NE 30 D, by BASF under the names Acronal® 601, Luhydran® LR 8833 or 8845, and by Hoechst under the names Appretan® N 9213 or N9212;
copolymers of acrylonitrile and of a non-ionic monomer chosen, for example, from butadiene and alkyl (meth)acrylates; mention may be made of the products provided under the names Nipol® LX 531 8 by Nippon Zeon and those provided under the name CJ 0601 8 by Rohm & Haas;
polyurethanes, such as the products provided under the names Acrysol® RM 1020 or Acrysol® RM 2020 by Rohm & Haas or the products Uraflex® XP 401 UZ and Uraflex® XP 402 UZ provided by DSM Resins;
copolymers of alkyl acetate and of urethane, such as the product 8538-33 provided by National Starch;
polyamides, such as the product Estapor® LO 11 provided by Rhône-Poulenc; and
chemically modified and unmodified non-ionic guar gums.

Unmodified non-ionic guar gums include, for example, the products sold under the name Vidogum® GH 175 by Unipectine and under the name Jaguar® C by Meyhall.

Modified non-ionic guar gums which can be used herein include, for example, modified with C₁-C₆ hydroxyalkyl groups. Mention may be made, for example, of the hydroxymethyl, hydroxyethyl, hydroxypropyl, and hydroxybutyl groups.

These guar gums are well known in the state of the art and can, for example, be prepared by reacting corresponding alkene oxides, such as propylene oxides, with guar gum, so as to obtain a guar gum modified with hydroxypropyl groups.

Such non-ionic guar gums, optionally modified with hydroxyalkyl groups, are, for example, sold under the trade names Jaguar® HP8, Jaguar® HP60, Jaguar® HP120, Jaguar® DC 293 and Jaguar® HP 105 by Meyhall and under the name Galactasol® 4H4FD2 by Aqualon.

The alkyl groups of the non-ionic polymers, for example, comprise from 1 to 6 carbon atoms.

The reducing agents can be chosen from thioacids and their salts (thioglycolic acid or thiosulphate, cysteine or cysteamine), alkali metal and alkaline earth metal sulphites, reducing sugars, such as glucose, vitamin C and its derivatives, sulphovinic (ethyl sulfuric) acid derivatives, and phosphines.

The coloring agents can be chosen from linear and aromatic (heterocyclic and nonheterocylic) conjugated structures. Mention may be made, for example, of nitrobenzene dyes, aromatic dyes, aminobenzene dyes, azo dyes, anthraquinone dyes, aromatic diamines, aminophenols, phenols and naphthols, porphyrins, tetraphenylporphyrins, metalloporphyrins, phthalocyanines, carotenoids, flavonoids or fluorescent molecules (fluorescein, rhodamine, coumarin, and the like).

The film-forming agents can be chosen from film-forming polymers, for example, those disclosed in French Patent Nos. 2 739 022, 2 757 048, and 2 767 699.

The foaming agents may be chosen from any art recognized surfactants with a foaming nature including cationic polymers and anionic polymers with foaming properties; or alternatively the foaming agent can be a specific agent, such as that disclosed in French Patent No. 2 751 221.

The particles, as cosmetic active principles, are other than the particles with a core-shell structure as disclosed herein and can be chosen from organic, inorganic, and composite particles.

The cosmetic compositions as disclosed herein are characterized by the particles which they comprise. These particles are, as disclosed herein, particles which can be defined as being metal nanoparticles encapsulated by an inorganic material.

As used herein, the term “nanoparticles” means particles with a size of less than or equal to 500 nm, such as ranging from 1 nm to 500 nm, for example, ranging from 1 nm and 100 nm, further, for example, from 1 nm to 50 nm.

As used herein, the term “particle size” means the maximum dimension which it is possible to measure between two points of the particle. Such sizes can be measured directly by microscopic techniques, such as scanning electron microscopy or atomic force microscopy, or by indirect techniques, such as dynamic light scattering.

The particles incorporated in the compositions as disclosed herein can have various shapes. They can, for example, assume a shape chosen from spheres, flakes, fibers, tubes, and polyhedra. They can also have an entirely random shape. In one aspect of the present disclosure, the particles are spherical.

The particles incorporated in the compositions as disclosed herein have a core composed of at least one metal. According to one embodiment, the core is predominately metal.

As disclosed herein, the term “metal” means a simple body composed solely of atoms of a metal element capable of generating cations.

As disclosed herein, the term “predominantly” means that the core of the particle is composed of 50% or more by weight of at least one metal.

For example, the core can be composed of at least 80% by weight, such as of at least 90% by weight and further, for example 100% by weight of at least one metal.

As disclosed herein, the term “metal” means aluminium and all the elements with an atomic number ranging from 21 to 82 and comprising Groups 3 to 13 of the Periodic Table of the Elements according to the new IUPAC notation: reference may be made, on this subject, to the CRC Handbook of Chemistry and Physics, 80th Print Edition.

As disclosed herein, the term “metal” also includes all the alloys of these elements, and the mixtures of these metals and alloys.

The core can thus also be composed, in the abovementioned percentages, of a mixture of at least two of these metals and/or alloys thereof.

The core can also be a composite core comprising several regions, wherein adjacent regions comprise different metals, alloys or mixtures thereof.

In one embodiment, the core may be a multilayer cores comprising an inner core forming a substrate comprising at least one of metals, alloys or mixtures thereof, at least partially covered by a first layer comprising at least one of metals, metal alloys or mixtures thereof which is different from that constituting the inner core, and optionally by at least one other layers, wherein each of these layers at least partially covers the preceding layer and each layer comprising at least one of metals, alloys or mixture of metals or alloys which is different from the following layer (if the latter exists) and from the preceding layer.

Apart from the at least one metal, the core can further comprise stabilizers of any kind and unavoidable impurities.

In addition, the core can also comprise, for example, metal compounds other than metals, such as metal oxides.

Thus, in the case of aluminium, the core can comprise alumina Al₂O₃, for example, in an amount of 10% by weight Al₂O₃ per 90% of Al metal.

The metal is, for example, chosen from transition metals, rare earth metals and their alloys and mixtures thereof.

In another aspect of the present disclosure, the metal is chosen from aluminium, copper, silver, gold, indium, iron, platinum, nickel, molybdenum, titanium, tungsten, antimony, palladium, zinc, tin, and mixtures thereof.

In the preceding list, the metals referred to as “noble” and copper can, for example, be used. As used herein, the term “noble metals” means gold, silver, palladium, platinum, and their alloys and mixtures thereof.

Silver, for example, can be used.

The solid shell can be in direct contact with the at least one metal of which the core comprises; in other words, no underlayer need be inserted between the solid shell and the at least one metal; or else the core comprises at least one metal which, before it is encapsulated or before the formation of the shell, can be modified at the surface by a treatment which modifies the properties of the latter. This treatment can comprise stabilizing the surface of the core (i.e., the surface of the metal) by an adsorbed or covalently bonded monolayer.

As disclosed herein, the shell surrounding the core (which may optionally be provided with a layer as described above) comprises of an inorganic material.

As disclosed herein, this material is a material which is solid at ambient temperature.

There exists no limitation with regard to the nature of the at least one inorganic material.

For example, the at least one inorganic material is chosen from materials composed of metal oxides and organometallic polymers.

The metal oxides are, for example, chosen from silicon, titanium, cerium, aluminium, zirconium, zinc, boron, lithium, magnesium, and sodium oxides, the mixed oxides of the latter, and the mixtures of these oxides and mixed oxides.

The metal oxides, which can be used herein, include, but are not limited to, silica, titanium oxide, and alumina.

The organometallic polymers are, for example, chosen from the products resulting from the polycondensation of alkoxysilanes.

The solid shell or the capsule comprising an inorganic material can have a thickness ranging from 2 nm to 300 nm, such as from 5 nm to 250 nm, further, for example, from 10 nm to 100 nm.

It should be noted that this solid shell or this capsule, and in accordance with the well-known definition of encapsulation in the technical field, is not a monolayer or a molecular layer but actually a layer which may be described as a “thick” wall, the thickness of which is generally within the range defined above.

As disclosed herein, the capsule, coating or shell is connected to the core by a physical bond, without covalent bonds. In other words, in the particles incorporated in the compositions as disclosed herein, the core/shell interface is defined as not exhibiting covalent bonds.

The shell or capsule around the metal core in the particles of the compositions as disclosed herein can be formed by various processes.

These processes, which are generally denoted by the terms of encapsulation or nanoencapsulation process, are known to a person skilled in the art in this technical field and can be generally divided into two main families: namely, on the one hand, physicochemical processes and, on the other hand, chemical processes.

The physicochemical processes can be chosen from phase separation and coacervation, controlled precipitation and any other known physicochemical process for microencapsulation.

The chemical processes can be chosen from interfacial polycondensation, in situ polycondensation, emulsion polymerization and any other known chemical process for microencapsulation.

For further details with regard to these encapsulation processes, reference may be made to the document “Microencapsulation Methods and Industrial Applications”, (ISBN 0-8247-9703-S).

For example, among the various encapsulation processes, encapsulation by a sol-gel process can be used.

The at least one inorganic material which comprises the shell of the particles is, for example, chosen from inorganic materials capable of being obtained by a sol-gel process, such as from metal oxides and organometallic polymers capable of being obtained by a sol-gel process from at least one precursor.

For example, the at least one inorganic material which comprises the shell of the particles can be chosen from metal oxides and organometallic polymers capable of being obtained or synthesized by polycondensation of at least one metal alkoxide precursor such as chosen from silicon, aluminium, boron, lithium, magnesium, titanium and zirconium alkoxides, and the mixed alkoxides thereof.

For further details with regard to the nature of the at least one precursor and the reaction mechanisms, reference may be made to the work “Sol-Gel Science”, edited by C. J. Brinker and G. W. Scherer and published by Academic Press (ISBN 0-12-134970-5).

Such a sol-gel process makes it possible to obtain a core-shell system comprising a metal core and a capsule of metal oxide or of organometallic polymer with a thickness generally of greater than 2 nm and conventionally ranging from 2 nm to 300 nm. For further details with regard to the sol-gel encapsulation process, reference may be made to the following papers:

“Synthesis and Self Assembly of Au/SiO₂ Core-Shell Colloids” (Nano Letters, 2002, 2 (7), 785-788).

“Cocondensation of Organosilica Hybrid Shells on Nanoparticle Templates: A Direct Synthetic Route to Functionalized Core-Shell Colloids” (Langmuir, 2000 16, 1454-1456).

“Synthesis and Characterization of Gold-Silica Nanoparticles Incorporating a Mercaptosilane Core-Shell Interface” (Langmuir, 2002, 18, 8566-8572).

The at least one precursor is, for example, alkoxysilanes.

As used herein, the term “alkoxysilane” means molecules comprising at least one, i.e., one, two or three, silicon atoms and at least two hydroxyl functional groups or at least two hydrolyzable functional groups, such as methoxy, ethoxy, propoxy, and the like. In addition, the alkoxysilane can optionally comprise functional groups which render it compatible with the physiologically acceptable medium, such as with a solvent of the latter, and/or which provide it with an affinity with keratinous substances or fibers. Mention may be made, among these functional groups, of the main functional groups which improve the solubility in water, such as the alkyl amine, alkyl alcohol, alkyl thiol, alkyl acid, alkyl polyamine, alkyl polyol and alkyl polycarboxyl functional groups. Mention may be made, among the water-soluble alkoxysilanes which can be used as the at least one precursor of the capsule, including, but not limited to, of 3-aminopropyltriethoxysilane, (3-aminopropyl)methyldiethoxysilane, {3-[bis(hydroxyethyl)amino]propyl}triethoxysilane, and the like.

Tetraethyl orthosilicate (TEOS) is, for example, among the at least one precursor of the shell or of the capsule.

In some embodiments, the coated or encapsulated particles included in the compositions as disclosed herein are capable of being prepared by a process comprising condensing, in an aqueous medium, water-soluble organic silicon compounds, which are optionally polymerized only to a slight extent and are chosen from organosilanes comprising a silicon atom and organosiloxanes comprising at least two silicon atoms, on the core, i.e., the particles are intended to form the core of the “coated” core-shell particles for example. The organic silicon compounds further comprise, in some embodiments, at least one basic chemical functional group and at least two hydrolysable or hydroxyl groups per molecule. The organic silicon compounds, which are not polymerized or which are polymerized only to a slight extent, are neutralized in an amount ranging from 1/1000 to 99/100, such as from 0.2/100 to 70/100, by a neutralizing agent. This process is described in French Patent No. 2 783 164.

In some other embodiments of the disclosure, the coated or encapsulated particles included in the compositions as disclosed herein are capable of being prepared by a process comprising condensing, in an aqueous medium, water-soluble organic silicon compounds, which are optionally polymerized only to a slight extent and which, in an embodiment, are chosen from organosilanes comprising a silicon atom and organosiloxanes comprising at least two silicon atoms, on the core, i.e., the particles being intended to form the core of the “coated” core-shell particle for example. The organic silicon compounds further comprise, per molecule, a group chosen from at least two hydroxyl groups or two hydrolysable functional groups and at least two non-hydrolysable functional groups, at least one of these non-hydrolysable functional groups having a cosmetic effect and at least one other of these non-hydrolysable functional groups is a solubilizing functional group. This process is described in French Patent No. 2 783 165.

For example, the at least one non-hydrolysable group having a cosmetic effect is a group having a function chosen from coloring, UV screening, bactericidal, fungicidal, and reducing functions.

In some embodiments, the coated or encapsulated particles included in the compositions as disclosed herein are capable of being prepared by a process comprising condensing, in an aqueous medium, water-soluble organic silicon compounds, which are optionally polymerized only to a slight extent and are chosen from organosilanes comprising a silicon atom and organosiloxanes comprising at least two silicon atoms, on the core, i.e., the particles are intended to form the core of the “coated” core-shell particle for example. The organic silicon compounds, in some embodiments, further comprise at least one non-basic solubilizing chemical functional group and at least two hydrolysable groups per molecule. This process is described in International Patent Application No. WO-A-01/22931.

In further embodiments, the coated or encapsulated particles included in the compositions as disclosed herein are capable of being prepared by a process comprising the following stages:
a) preparing a mixture of
   (i) a dispersion in a liquid medium, for example, chosen from an aqueous and alcoholic and oily medium, optionally in the presence of a dispersant, of particles comprising at least one metal and for example, intended to form the core of the core-shell particle,
   (ii) and of a solution of a crosslinked hybrid organic/inorganic material, the material (before hydrolysis) is obtained by the sol-gel route from a premix comprising:
      (A) at least one compound chosen from metal and organometallic compounds, and
      (B) at least one polymer chosen from functionalized organic polymer or precursor of this polymer and functionalized silicone polymer or a precursor of this polymer, the latter being different from (A),
b) contacting the mixture with water when the dispersion of the particles intended to form the core is a dispersion in an alcoholic or oily liquid medium.

The hybrid organic/inorganic material used for the process which makes it possible to obtain the solid shell of the particles of the composition as disclosed herein in this embodiment is known and has been described, for example, in French Patent Application No. 97 04157 and in French Patent Application No. FR-A-2 825 917.

The outer surface of the particles, i.e., the outer surface of the capsule or of the shell, can be covalently modified by at least one chemical group that is capable of improving the adsorption of the particles on keratinous substances, such as hair. The ester surface of the particles, i.e., the outer surface of the capsule or of the shell of the particles, can also be covalently modified by at least one chemical group that is capable of reacting chemically with keratinous substances, such as hair.

In the first case, the adsorption on keratinous substances, such as hair, of the core-shell nanoparticles of the compositions as disclosed herein can be improved by covalently modifying the capsule of organic material, such as a polymer, with various chemical groups (Group A below) which render the surface of the particles, for example, more hydrophobic and/or more cationic and/or more anionic and/or more hydrophilic.

The adsorption is defined as employing lower bonding energies than covalent bonds, i.e., less than 50 kcal/mol, between the keratinous substance, such as the individual hair, and the particle. These low-energy bonds are, for example, Van der Waals forces, hydrogen bonds, electron donor-acceptor complexes, and the like.

The group capable of improving the adsorption of the particles on keratinous substances is generally chosen from the groups of the following Group A:

Group A:
Carboxylic acids and their salts,
Primary, secondary, tertiary and quaternary amines,
Phosphates,
Sulphur oxides, such as sulphones, sulphonic, sulphoxides and sulphates,
Aromatic rings, such as phenyl, triazine, thiophene and imidazole.

In the second case, it is also possible to promote the adhesion to keratinous substances, such as the hair, of the nanoparticles as disclosed herein by covalently modifying the capsule of inorganic material with various groups (Group B) capable of reacting chemically with the keratinous substance. For example, the term “groups having a reactivity with regard to the keratinous substance, such as the individual hair,” means the groups capable of forming a covalent bond with this substance, for example with the amines and/or the carboxylic acids and/or the thiols of the amino acids constituting the keratinous substance. The formation of these covalent bonds can either be spontaneous or can be carried out by activation by temperature, pH, light, a co-reactant or a chemical or biochemical catalyst, such as an enzyme.

The group capable of reacting chemically or able to react chemically with keratinous substances, such as hair, is generally chosen from the groups of the following Group B:

Group B:
Epoxides,
Vinyl and activated vinyl: such as acrylonitrile, acrylic and methacrylic esters, crotonic acid and esters, cinnamic acid and esters, styrene and derivatives, butadiene, vinyl ethers, vinyl ketones, maleic esters, maleimides, vinyl sulphones, and the like,
Carboxylic acids and their derivatives: such as anhydride, acid chloride, esters,
Acetals, hemiacetals,
Aminals, hemiaminals,
Ketones and α-hydroxyketones, α-haloketones,
Lactones, thiolactones,
Isocyanates,
Thiocyanates,
Imines,
Imides (such as succinimides, glutimides),
Pyridyldithio,
N-Hydroxysuccinimide esters,
Imidates,
Oxazine and oxazoline,
Oxazinium and oxazolinium,
Groups of formula R₁X wherein R₁ is a group chosen from C₁ to C₃₀ alkyl groups, C₆ to C₃₀ aryl groups and C₇ to C₃₀ aralkyl groups (wherein the alkyl group is chosen from C₁ to C₃₀) and X is a leaving group such as I, Br, Cl, OSO₃R, wherein R is chosen from H and C₁ to C₃₀ alkyl groups, —SO₂R′, where R′ is chosen from H or C₁ to C₃₀ alkyl groups, tosyl groups, N(R″)₃, wherein R″ is chosen from C₁ to C₃₀ alkyl groups, and OPO₃R′″₂, wherein R′″ is chosen from H and C₁ to C₃₀ alkyl groups; for example, groups of formula R₁X can be chosen from alkyl, aryl, and aralkyl halides;
Groups of formula R₂X wherein R₂ is chosen from carbon rings of C₃ to C₃₀ and unsaturated heterocycles with 3 to 20 ring members comprising at least one heteroatom chosen from N, S, O and P, and X is a leaving group as defined above; for example, formula R₂X can be chosen from the halides of unsaturated rings, such as chlorotriazine, chloropyrimidine, chloroquinoxaline and chlorobenzotriazole,
Groups of formula R₃SO₂X, wherein R₃ has the same meaning as R₁ and X is a leaving group and has the meaning already given above,
Lactones,
Thiolactones, and
Siloxanes.

Mention may be made, by way of example, without implied limitation, of the activation by N-hydroxysulphosuccinimide of core-shell particles having a core made of silver and a capsule made of titanium oxide, which capsule is obtained by polycondensation of titanium tetraethoxide (tetraethoxytitanate). The sulphosuccinimide groups grafted to the surface of the nanoparticles of the compositions as disclosed herein make it possible to covalently bond the nanoparticles of the compositions as disclosed herein to the hair by reaction with the free surface amines which the hair fiber possesses.

For further details with regard to this activation process, reference may be made to the following document: “Biofunctionalised Biocompatible Titania Coatings for Implants”, Key Eng. Mat., 206-213 (2002), 1547-1550.

It should be noted that the chemical functional groups on the surface of the keratinous substance, for example, of the hair fiber, can be increased in density by pretreatment of the fiber with a solution of polymer having a particular affinity for the fiber and exhibiting reactive functional groups. In the preceding example, the density of the amine functional groups at the surface of the fiber can be increased, for example, by absorbing polyethyleneimine beforehand.

In order to increase the durability of the effect over time, in addition to the improvement in the adhesion and/or in the adsorption, it is possible to use metal particles encapsulated by a shell of reactive organometallic polymer capable of creating interparticle covalent bonds after evaporation of the solvent phase.

In this context, mention may be made, without implied limitation, of the encapsulation of particles by a polymethacrylate having alkoxysilane functional groups.

For further details with regard to this process, reference may be made to the following document “Synthesis and characterization of SiOH functionalized polymer latexes using methacryloxypropyltrimethoxysilane in emulsion polymerisation” (Macromolecules, 2002, 35, 6185-6191).

In the cosmetic compositions, the encapsulated metal nanoparticles as disclosed herein are generally present in an amount ranging from 0.0001% to 50%, such as from 0.01% to 5% and further, for example, from 0.05% to 2%, by weight relative to the total weight of the composition.

The composition as disclosed herein additionally comprises a physiologically acceptable medium. As disclosed herein, the term “physiologically acceptable medium” means a medium capable of being applied to keratinous fibers such as to the hair of human beings.

The physiologically acceptable medium of the composition generally comprises at least one solvent. The at least one solvent makes it possible, for example, to convey the encapsulated metal nanoparticles. The at least one solvent can be chosen generally from organic solvents, water, and mixtures thereof.

The organic solvents are generally chosen from C₁ to C₄ aliphatic alcohols, such as ethanol and isopropanol, polyols, such as glycerol and propylene glycol, aromatic alcohols, such as benzyl alcohol, alkanes, for example C₅ to C₁₀ alkanes, acetone, methyl ethyl ketone, methyl acetate, butyl acetate, alkyl acetate, dimethoxyethane, diethoxyethane and their mixtures.

The compositions as disclosed herein can be packaged in various forms such as in an aerosol device.

The composition as disclosed herein can further comprise at least one propellant. The at least one propellant comprises compressed or liquefied gases commonly employed for the preparation of aerosol compositions. For example, air, carbon dioxide gas, compressed nitrogen and a soluble gas, such as dimethyl ether, halogenated (e.g., fluorinated) and nonhalogenated hydrocarbons, and their mixtures can be used.

The compositions as disclosed herein further comprise conventional cosmetic additives chosen from one or more of, for example, reducing agents, oxidizing agents, thickening agents, softeners, antifoaming agents, direct and oxidation dyes, fragrances, peptizing agents, preservatives, anionic and amphoteric surfactants, and the like.

The cosmetic composition as disclosed herein can be a cosmetic treatment composition, such as a composition for contributing sheen to keratinous substances. In further embodiments, it is a hair cosmetic composition, such as a composition for contributing sheen to the hair.

The hair cosmetic compositions as disclosed herein, after application to the hair, can be rinsed out or left in. The compositions, such as hair compositions, (formulations) can be provided in various dosage forms chosen from lotions, sprays, foams, lacquers, conditioners, and shampoos.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The disclosure will be better understood with the help of the non-limiting illustrative examples which follow. In the examples, the percentages are expressed by weight and a.m. means active material.

### COMPARATIVE EXAMPLE

In this example, a composition as disclosed herein and a composition according to the prior art were prepared.

Composition 1: Aerosol Foam in Accordance With the Present Disclosure

| Starch acetate | 5% a.m. |
|---|---|
| Polysorbate 20 | 0.1% a.m. |
| Cocamidopropyl betaine | 0.5% a.m. |
| Encapsulated silver nanoparticles^{[1]} | 1.0% a.m. |
| Laureth-4 | 0.3% a.m. |
| Isobutane/butane/propane | 5% a.m. |
| Preservative | q.s. |
| Fragrance | q.s. |
| Water | q.s. for |
| Polysorbate 20: Polyoxyethylene (20) sorbitan monolaurate, sold by Atlas. | |
| Laureth-4 surfactant: sold by Uniquema. | |
| Butane/isobutane/propane mixture: 24/56/20. | |
| ^{[1]}The core-shell nanoparticles were synthesized according to the process described in Example No. 3 of International Patent Application No. WO-A-01/88540. The silver core of the particle was obtained by reduction of silver nitrate (AgNO₃) by sodium | |
| # borohydride (NaBH₄). For its part, the encapsulation was carried out by emulsion polycondensation of tetraethyl orthosilicate (TEOS). | |

As was shown by the transmission electron microscopy (TEM) exposures taken, the particles thus encapsulated exist in the form of a native silver core with a diameter ranging from 5 nm to 30 nm covered by a silica layer of approximately 5 nm.

Composition 2: Control Aerosol Foam Not in Accordance With the Disclosure

| | |
|---|---|
| Starch acetate | 5% a.m. |
| Polysorbate 20 | 0.1% a.m. |
| Cocamidopropyl betaine | 0.5% a.m. |
| Non-encapsulated silver nanoparticles^{[2]} | 1.0% a.m. |
| Laureth-4 | 0.3% a.m. |
| Isobutane/butane/propane | 5% a.m. |
| Preservative | q.s. |
| Fragrance | q.s. |
| Water | q.s. for |

Polysorbate 20: Polyoxyethylene (20) sorbitan monolaurate, sold by Atlas.
Laureth-4 surfactant: sold by Uniquema.
Butane/isobutane/propane mixture: 24/56/20.
^{[2]}Silver nanoparticles sold under the reference “Colloid Mag” by Grant Industries. As was shown by the transmission electron microscopy (TEM) exposures taken, the nanoparticles exhibited a diameter ranging from 5 nm to 30 nm.

Each of the preceding compositions was applied to a lock of brown hair weighing 2.7 g (European hair with a length of 20 cm) at the rate of one gram of composition per lock. After application, the locks were dried under a hairdryer (70° C.) for 30 minutes.

A measurement of sheen was subsequently carried out on a batch of 10 locks treated as indicated above with the compositions of the present disclosure and those not in accordance with the present disclosure.

The sheen was determined using a photogoniometer by measuring the specular and diffuse reflections of the locks of hair laid flat on a support. Using a 175 watt xenon arc lamp (model ORC175F) coupled to a V filter (lambda), light was emitted over the lock under an angle of +30° with respect to the normal to its surface. Using a movable receiving arm, the specular reflection (R), corresponding to the maximum light intensity reflected in the vicinity of an angle of −30°, and the diffuse reflection (D), corresponding to the light reflected at an angle of +15°, were measured. According to the disclosure, the sheen was determined by calculating the ratio (R)/(D).

In order to evaluate the retention of the sheen over time, the measurement of sheen was repeated on the same treated locks after storing the treated locks for one month under ambient conditions (20° C. and 50% RH).

The results obtained in terms of stability of the sheen are given in the following table:

TABLE

| | Sheen | | |
|---|---|---|---|
| | before | Sheen immediately | Sheen of the treated |
| | treatment | after treatment | locks after 1 month |
| Composition 1 | 23 ± 3 | 37 ± 1 | 34 ± 3 |
| (invention) | | | |
| Composition 2 | 20 ± 4 | 33 ± 4 | 20 ± 2 |
| (prior art) | | | |

As shown in the preceding table, it is recorded that the composition in accordance with the disclosure retains these sheen properties after storage of the locks under ambient conditions for 1 month. By way of comparison, the composition not in accordance with the disclosure lost its reflectivity after storage of the locks under ambient conditions for 1 month.

In addition to the retention of the sheen over time, the cosmetic compositions as disclosed herein can exhibit a better stability over time with respect to the state of the art. This can be due, in part, to the fact that encapsulation limits the aggregation of the metal nanoparticles in polar media, such as water and/or ethanol, thus making it possible to obtain colloidal dispersions of high stability.

1. A cosmetic composition comprising, in a physiologically acceptable medium,
at least one agent exhibiting a cosmetic activity and
particles comprising a core and a solid shell bonded to the core via a noncovalent bond, wherein the core comprises at least one metal, the solid shell comprises at least one inorganic material, and the size of the particles is less than or equal to 500 nm.

2. The composition according to claim 1, wherein the at least one agent exhibiting a cosmetic activity is chosen from:
saccharides, oligosaccharides and polysaccharides, which may be optionally hydrolyzed and/or modified,
amino acids, oligopeptides, peptides, proteins, which may be optionally hydrolysed and/or modified, poly(amino acid)s and enzymes,
branched and unbranched fatty acids and alcohols,
animal, vegetable and mineral waxes,
ceramides and pseudoceramides,
hydroxylated organic acids,
UV screening agents,
antioxidants and agents for combating free radicals,
chelating agents,
antidandruff agents,
seborrhoea-regulating agents,
soothing agents,
cationic surfactants,
cationic and amphoteric polymers,
organomodified and nonorganomodified silicones,
mineral, vegetable and animal oils,
polyisobutenes and poly(α-olefin)s,
esters,
soluble and dispersed anionic polymers,
soluble and dispersed non-ionic polymers,
reducing agents,
coloring agents and coloring materials,
foaming agents,
film-forming agents,
particles, other than the particles comprising the core and the solid shell bonded to the core via a noncovalent bond,
and mixtures thereof.

3. The composition according to claim 2, wherein the coloring agents and coloring materials are chosen from hair dyes.

4. The composition according to claim 1, wherein the at least one agent exhibiting a cosmetic activity is present in an amount ranging from 0.001% to 10%, by weight relative to the total weight of the cosmetic composition.

5. The composition according to claim 4, wherein the at least one agent exhibiting a cosmetic activity is present in an amount ranging from 0.01% to 5%, by weight relative to the total weight of the cosmetic composition.

6. The composition according to claim 1, wherein the size of the particles ranges from 1 nm to 500 nm.

7. The composition according to claim 6, wherein the size of the particles ranges from 1 nm and 100 nm.

8. The composition according to claim 7, wherein the size of the particles ranges from 1 nm to 50 nm.

9. The composition according to claim 1, wherein the particles have a shape chosen from spheres, flakes, fibers, tubes, polyhedra, and random shapes.

10. The composition according to claim 1, wherein the core of the particles comprises at least 80% by weight of the at least one metal.

11. The composition according to claim 10, wherein the core of the particles comprises at least 90% by weight of the at least one metal.

12. The composition according to claim 11, wherein the core of the particles comprises 100% by weight of the at least one metal.

13. The composition according to claim 1, wherein the at least one metal is chosen from aluminium and elements with an atomic number ranging from 21 to 82 and in Groups 3 to 13 of the Periodic Table of the Elements, and alloys thereof.

14. The composition according to claim 13, wherein the core of the particles comprises a mixture of at least two of the metals and/or alloys thereof.

15. The composition according to claim 1, wherein the core of the particles is a composite core comprising at least two regions, and adjacent regions comprise different metals, alloys and/or mixtures thereof.

16. The composition according to claim 15, wherein the composite core is a multilayer composite core comprising an inner core comprising at least one of metals, alloys and mixtures thereof, the inner core is at least partially covered by a first layer comprising at least one of metals, metal alloys and mixture thereof which is different from that of the inner core and optionally by at least one other layer, wherein each of these layers at least partially covering the preceding layer and each of these layers comprising at least one of metals, alloys and mixtures thereof, which is different from the following layer and from the preceding layer.

17. The composition according to claim 1, wherein the core of the particles further comprises at least one stabilizer.

18. The composition according to claim 1, wherein the core of the particles further comprises at least one metal oxide.

19. The composition according to claim 1, wherein the at least one metal is chosen from transition metals, rare earth metals, and their alloys.

20. The composition according to claim 1, wherein the at least one metal is chosen from aluminium, copper, silver, gold, indium, iron, platinum, nickel, molybdenum, titanium, tungsten, antimony, palladium, zinc, tin, and their alloys.

21. The composition according to claim 20, wherein the at least one metal is chosen from gold, silver, palladium, platinum, and their alloys.

22. The composition according to claim 21, wherein the at least one metal is silver.

23. The composition according to claim 1, wherein the solid shell is in direct contact with the at least one metal.

24. The composition according to claim 1, wherein the core is modified at the surface by a treatment wherein the treatment modifies the properties of the core.

25. The composition according to claim 24, wherein the treatment comprises stabilizing the surface of the core by an adsorbed or covalently bonded monolayer.

26. The composition according to claim 1, wherein the at least one inorganic material is chosen from materials comprising at least one metal oxide and organometallic polymers.

27. The composition according to claim 26, wherein the at least one metal oxide is chosen from silicon, titanium, aluminium, zirconium, zinc, boron, lithium, magnesium, sodium and cerium oxides, mixed oxides of sodium and cerium oxides, and mixtures of sodium and cerium oxides, and mixed oxides thereof.

28. The composition according to claim 27, wherein the metal oxide is chosen from silica, titanium oxide and alumina.

29. The composition according to claim 26, wherein the organometallic polymers are chosen from the condensation products of alkoxysilanes.

30. The composition according to claim 1, wherein the solid shell has a thickness ranging from 2 nm to 300 nm.

31. The composition according to claim 30, wherein the solid shell has a thickness ranging from 5 nm to 250 nm.

32. The composition according to claim 31, wherein the solid shell has a thickness ranging from 10 nm to 100 nm.

33. The composition according to claim 1, wherein the solid shell is formed by a physicochemical process chosen from phase separation, and coacervation and controlled precipitation.

34. The composition according to claim 1, wherein the solid shell is formed by a chemical process chosen from interfacial polycondensation, in situ polycondensation, and emulsion polymerization.

35. The composition according to claim 1, wherein the at least one inorganic material is chosen from inorganic materials capable of being obtained by a sol-gel process.

36. The composition according to claim 35, wherein the at least one inorganic material is chosen from metal oxides and organometallic polymers capable of being obtained by a sol-gel process from at least one precursor.

37. The composition according to claim 35, wherein the at least one inorganic material is chosen from at least one of metal oxides and organometallic polymers capable of being obtained by polycondensation of at least one metal alkoxide precursor.

38. The composition according to claim 37, wherein the at least one metal alkoxide precursor is chosen from silicon, aluminium, boron, lithium, magnesium, titanium and zirconium alkoxides.

39. The composition according to claim 36, wherein the organometallic polymers are chosen from organometallic polymers capable of being prepared by polycondensation of alkoxysilanes comprising at least one silicon atom and at least two functional groups chosen from hydroxyl and hydrolysable functional groups, and optionally comprising at least one additional functional group that renders compatible with the physiologically acceptable medium and/or contributes an affinity with keratinous substances or fibers.

40. The composition according to claim 39, wherein the hydroxyl and hydrolyzable functional groups are chosen from methoxy, ethoxy, and propoxy.

41. The composition according to claim 39, wherein the at least one additional functional group is chosen from functional groups that improve the oragnometallic polymers solubility in water.

42. The composition according to claim 41, wherein the at least one additional functional group is chosen from alkyl amine, alkyl alcohol, alkyl thiol, alkyl acid, alkyl polyamine, alkyl polyol and alkyl polycarboxyl functional groups.

43. The composition according to claim 39, wherein the alkoxysilanes are chosen from 3-aminopropyltriethoxysilane, (3-aminopropyl)methyldiethoxysilane and {3-[bis(hydroxyethyl)amino]propyl}triethoxysilane.

44. The composition according to claim 36, wherein the precursor of the sol-gel process is tetraethyl orthosilicate.

45. The composition according to claim 1, wherein the particles are capable of being prepared by a process comprising condensing, in an aqueous medium, water-soluble organic silicon compounds, which are optionally polymerized to a slight extent and chosen from organosilanes comprising a silicon atom and organosiloxanes comprising at least two silicon atoms, to form the core, and the organic silicon compounds are neutralized in an amount ranging from 1/1000 to 99/100, by a neutralizing agent.

46. The composition according to claim 45, wherein the organic silicon compounds further comprise at least one basic chemical functional group and at least two hydrolyzable and/or hydroxyl groups per molecule.

47. The composition according to claim 45, wherein the organic silicon compounds are neutralized in an amount ranging from 0.2/100 to 70/100, by the neutralizing agent.

48. The composition according to claim 1, wherein the particles are capable of being prepared by a process comprising condensing, in an aqueous medium, water-soluble organic silicon compounds, which are optionally polymerized to a slight extent and chosen from organosilanes comprising a silicon atom and organosiloxanes comprising at least two silicon atoms, to form the core, and the organic silicon compounds further comprise, per molecule, at least two hydroxyl groups or two hydrolysable functional groups and at least two non-hydrolysable functional groups, at least one of the non-hydrolysable functional groups has a functional group having a cosmetic effect and at least one other of these non-hydrolysable functional groups has a solubilizing functional group.

49. The composition according to claim 48, wherein the cosmetic effect is at least one function chosen from coloring, UV screening, bactericidal and fungicidal, and reducing functions.

50. The composition according to claim 1, wherein the particles are capable of being prepared by a process comprising condensing, in an aqueous medium, water-soluble organic silicon compounds, which are optionally polymerized to a slight extent and are chosen from organosilanes comprising a silicon atom and organosiloxanes comprising two or three silicon atoms, to form the core, and the organic silicon compounds further comprise at least one non-basic solubilizing chemical functional group and at least two hydrolysable groups per molecule.

51. The composition according to claim 1, wherein the particles are prepared by a process comprising:
a) preparing a mixture of
(i) a dispersion in a liquid medium, the liquid medium chosen from aqueous, alcoholic, and oily medium, optionally in the presence of a dispersant, of particles comprising at least one metal wherein the core is formed, and
(ii) a solution of a crosslinked hybrid organic/inorganic material, wherein the crosslinked hybride material is obtained by a sol-gel route from a premix comprising:
(A) at least one compound chosen from metal and organometallic compounds, and
(B) at least one polymer chosen from functionalized organic polymer and a precursor thereof and at least one functionalized silicone polymer and a precursor thereof, the latter being different from (A), and
b) contacting the mixture with water when the dispersion of the particles is in the liquid medium chosen from alcoholic and oily medium.

52. The composition according to claim 1, wherein the particles have an outer surface that is covalently modified by at least one chemical group that is capable of improving the adsorption of the particles on a keratinous substance.

53. The composition according to claim 52, wherein the keratinous substance is hair.

54. The composition according to claim 1, wherein the particles have an outer surface that is covalently modified by at least one chemical group that is capable of reacting chemically with a keratinous substance.

55. The composition according to claim 54, wherein the keratinous substance is hair.

56. The composition according to claim 52, wherein the at least one chemical group is chosen from:
carboxylic acids and salts thereof,
primary, secondary, tertiary and quaternary amines,
phosphates,
sulphur oxides, and
aromatic rings.

57. The composition according to claim 56, wherein the sulphur oxides are chosen from sulphones, sulphonic, sulphoxides, and sulphates.

58. The composition according to claim 56, wherein the aromatic rings are chosen from phenyl, triazine, thiophene, and imidazole.

59. The composition according to claim 56, wherein the at least one chemical group is chosen from:
epoxides,
vinyl and activated vinyl,
earboxylic acids and derivatives thereof,
acetals, hemiacetals,
aminals, hemiaminals,
ketones and α-hydroxyketones, α-haloketones,
lactones, thiolactones,
isocyanates,
thiocyanates,
imines,
imides,
pyridyidithio,
N-hydroxysuccinimide esters,
imidates,
oxazine and oxazoline,
oxazinium and oxazolinium,
groups of formula R₁X wherein R₁ is chosen from C₁-C₃₀ alkyl groups, C₆-C₃₀ aryl groups, and C₇-C₃₀ aralkyl groups with the alkyl group chosen from C₁-C₃₀ alkyl groups and X is a leaving group,
groups of formula R₂X wherein R₂ is chosen from C₃-C₃₀ carbon ring and unsaturated heterocycles with 3 to 20 ring members comprising at least one heteroatom chosen from N, S, O and P, and X is a leaving group,
groups of formula R₃SO₂X, wherein R₃ chosen from C₁-C₃₀ alkyl groups, C₆-C₃₀ aryl groups, and C₇-C₃₀ aralkyl groups with the alkyl group chosen from C₁-C₃₀ alkyl groups and X is a leaving group,
lactones,
thiolactones, and
siloxanes.

60. The composition according to claim 59, wherein the vinyl and activated vinyl are chosen from acrylonitrile, acrylic and methacrylic esters, crotonic acid and esters, cinnamic acid and esters, styrene and derivatives, butadiene, vinyl ethers, vinyl ketones, maleic esters, maleimides, and vinyl sulphones.

61. The composition according to claim 59, wherein the carboxylic acids and derivatives thereof are chosen from anhydride, acid chloride, and esters.

62. The composition according to claim 59, wherein the imides are chosen from succinimides and glutimides.

63. The composition according to claim 59, wherein the leaving group in the groups of formulae R₁X, R₂X, and R₃SO₂X is chosen from I, Br, Cl, OSO₃R, wherein R is chosen from H and C₁-C₃₀ alkyl groups, —SO₂R′, wherein R′ is chosen from H and C₁-C₃₀ alkyl groups, a tosyl group, N(R″)₃, wherein R″ is C₁-C₃₀ alkyl groups, and OPO₃R′″₂, wherein R′″ is chosen from H and C₁-C₃₀ alkyl groups.

64. The composition according to claim 59, wherein the groups of formula R₁X are chosen from alkyl, aryl and aralkyl halides.

65. The composition according to claim 59, wherein groups of formula R₂X are halides of unsaturated rings chosen from chlorotriazine, chloropyrimidine, chloroquinoxaline, and chlorobenzotriazole.

66. The composition according to claim 1, wherein the solid shell of the particles comprises at least one reactive organometallic polymer that is capable of creating interparticle covalent bonds.

67. The composition according to claim 1, wherein the particles are present in an amount ranging from 0.0001% to 50%, by weight relative to the total weight of the composition.

68. The composition according to claim 67, wherein the particles are present in an amount ranging from 0.01% and 5%, by weight relative to the total weight of the composition.

69. The composition according to claim 68, wherein the particles are present in an amount ranging from 0.05% and 2%, by weight relative of the total weight of the composition.

70. The composition according to claim 1, wherein the physiologically acceptable medium comprises at least one solvent.

71. The composition according to claim 70, wherein the at least one solvent is chosen from organic solvents, water, and mixtures thereof.

72. The composition according to claim 71, wherein the organic solvents are chosen from at least one of C₁ to C₄ aliphatic alcohols, polyols, aromatic alcohols, alkanes, acetone, methyl ethyl ketone, methyl acetate, butyl acetate, alkyl acetate, dimethoxyethane, and diethoxyethane.

73. The composition according to claim 72, wherein the C₁ to C₄ aliphatic alcohols are chosen from ethanol and isopropanol.

74. The composition according to claim 72, wherein the polyols are chosen from glycerol and propylene glycol.

75. The composition according to claim 72, wherein the aromatic alcohols are benzyl alcohol.

76. The composition according to claim 72, wherein the alkanes are chosen from C₅ to C₁₀ alkanes.

77. The composition according to claim 1, further comprising at least one cosmetic additive chosen from reducing agents, oxidizing agents, thickening agents, softeners, antifoaming agents, direct and oxidation dyes, fragrances, peptizing agents, preservatives, and anionic and amphoteric surfactants.

78. The composition according to claim 1, wherein the composition is chosen from a cosmetic treatment composition for contributing sheen to keratinous substances.

79. The composition according to claim 78, wherein the composition is a hair composition for contributing sheen to the hair.

80. The composition according to claim 78, wherein the composition is in a form chosen from lotions, sprays, foams, lacquers, conditioners, and shampoos.

81. The composition according to claim 1, wherein the composition is packaged in an aerosol device.

82. A cosmetic process for the treatment of keratinous substances, comprising applying to keratinous substances a composition comprising, in a physiologically acceptable medium,
at least one agent exhibiting a cosmetic activity and
particles comprising a core and a solid shell bonded to the core via a noncovalent bond, wherein the core comprises at least one metal, the solid shell comprises at least one inorganic material, and the size of the particles is less than or equal to 500 nm.

83. The composition according to claim 82, wherein the keratinous substance is hair.

84. The composition according to claim 82, wherein the process is for contributing sheen to the keratinous substances.

85. The composition according to claim 84, wherein the keratinous substances is hair.

86. A process of making a cosmetic composition, comprising adding in a composition particles comprising a core and a solid shell bonded to the core via a noncovalent bond, wherein the core comprises at least one metal, the solid shell comprises at least one inorganic material, and the size of the particles is less than or equal to 500 nm, wherein the composition comprises, in a physiologically acceptable medium, at least one agent exhibiting a cosmetic activity and the resulting composition is for contributing sheen to keratinous substances.

87. The composition according to claim 86, wherein the keratinous substance is hair.
